(19) United States
(12) Patent Application Publication
(10) Pub. No.: US 2005/0055102 A1
(43) Pub. Date: Mar. 10, 2005

(54) Set of prosthetic elements for a tibial prosthetic assembly

(76) Inventors: Alain Tornier, Saint Ismier (FR); David Dejour, Lyon (FR); Michel Bonnin, Francheville (FR)

Correspondence Address: Ralph A. Dowell of DOWELL & DOWELL P.C., 2111 Eisenhower Ave., Suite 406, Alexandria, VA, 22314, US

(21) Appl. No.: 10/842,631

(22) Filed: May 11, 2004

(30) Foreign Application Priority Data
May 12, 2003 (FR) 03 05683

(51) Int. Cl. A61F 2/38

(52) U.S. Cl. 623/20.32; 623/20.15

(57) ABSTRACT

A tibial prosthetic assembly generally comprises a base (6) intended to be connected to a tibia and a shoe adapted to be fixedly mounted on the base. This shoe defines a prosthetic articulating surface intended to cooperate with a femoral element (2) and presenting an anterior spine (56) on either side of which are located two glenoidal cavities (54, 55) for receiving femoral condyles (14, 15).

In order to form such an assembly, the surgeon has at his disposal a set comprising the base (6) and a plurality of shoes which are adapted to be assembled in respective inherent angular positions in each of which the longitudinal axis of the anterior spine (56) of each shoe forms a predetermined angle with the antero-posterior median axis of the base (6). Application to the domain of prostheses of the knee joint, particularly of humans.

The present invention relates to a set of prosthetic elements for a tibial prosthetic assembly, which tibial assembly comprises a base intended to be connected to a tibia, as well as a shoe adapted to be mounted on the base and defining a prosthetic articulating surface intended to cooperate with a femoral prosthetic element.

It is applied to the domain of partial or complete prostheses of the knee joint, particular of humans.

Two large families of tibial prosthetic assemblies exist, depending on whether the shoe, mounted on the base, is mobile or not, particularly in rotation, with respect to the base. If the shoe is fixed with respect to the base, it is question of single-jointed tibial assemblies, i.e. assemblies allowing the articulation between solely the shoe and the femoral element, while, if the shoe is mobile with respect to the base, it is question of double-jointed tibial assemblies or of rotatory assemblies.

The invention concerns prosthetic elements for forming a single-jointed tibial assembly.

In order to put in place such a tibial assembly, the surgeon must firstly, in manner known per se, connect the base firmly to the upper end of the tibia. Then the shoe is fixed to the base, for example by clipping, screwing and/or gluing, in a pre-determined position with respect to the base.

However, it has proved from surgical practice that, depending on the particular anatomy of each patient and/or depending on which knee, the right or left, is operated, the position of the shoe imposed by the base and the femoral component is rarely the best adapted, i.e. the one allowing the patient to recover a behaviour of the joint close to the anatomical behaviour. Moreover, it is one of the reasons for which double-jointed prosthetic assemblies mentioned above have developed, but which remain more expensive and are mechanically less stable.

In order to allow a certain freedom of positioning of the shoe during putting into place with respect to the base, it has been proposed in the past to interpose between the base and the shoe shims of particular geometry. However, this solution is complex for the surgeon to carry out due to the large number both of constituents of the prosthetic assembly and of operating gestures necessary in order, at the end of the operation, to fix the relative positions of these constituents.

The purpose of the invention is to propose a set of prosthetic elements to form a single-jointed tibial prosthetic assembly, which makes it possible to obtain a tibial assembly which is more compact and more in conformity with the patient's anatomy, without for all that complicating the surgeon's work of putting in place, nor significantly increasing the cost of this assembly.

To that end, the invention has for its object a set of prosthetic elements for a tibial prosthetic assembly, comprising a base intended to be connected to the upper end of a tibia and defining a median antero-posterior axis, said tibial assembly comprising said base and a shoe adapted to be fixedly mounted on the base and defining a tibial prosthetic articulating face intended to cooperate with a femoral prosthetic element. This articulating surface presents both two glenoidal cavities for receiving internal and external condyles of the femoral element and an anterior spine which longitudinally defines an axis on either side of which are located these glenoidal cavities. This set comprises a plurality of shoes adapted to be fixedly mounted on the base in respective angular positions in each of which the axis of the anterior spine of each shoe forms, with the median axis of the base, a predetermined angle, at least one of these predetermined angles being non-zero and another possibly being zero.

The surgeon, having at his disposal the base of such a set and choosing a shoe from this set, may thus put in place a prosthetic assembly best suited to the patient's anatomy, while possibly correcting a poor angular positioning of the base with respect to the tibia.

According to other characteristics of this set, taken separately or in all technically possible combinations:
the shoes of the set are chosen from shoes of which the axes of the spines form with the median axis of the base angles whose respective values are included between about 0 and 15°.
the angles formed between the axes of the spines of the shoes of the set and the median axis of the base are respectively worth about 0, 2, 4, 6 and 8°.
each shoe of the set is provided with a face for assembly opposite the articulating surface, said face bearing means for fixing on the tibial base adapted to cooperate with this base by complementarity of shapes.
the assembly faces of the shoes of the set are substantially identical to one another.
the means for fixing each shoe of the set comprises a lip for clipping in a substantially complementary groove borne by the base.
the base comprises a peripheral flange and the peripheral contour of the assembly face of each shoe of the set is adapted to be inscribed inside the flange of the base.
at least corresponding parts of the interior contour of the flange and of the peripheral contour of the assembly face of each shoe of the set are adapted to cooperate by complementarity of shapes as fixing means; and
the articulating surface of each shoe of the set presents a third glenoidal cavity extending along the axis of the anterior spine and intended to receive a third condyle of the femoral element.

The invention also relates to a process for putting in place a tibial prosthetic assembly for a knee joint, in which:
a set of prosthetic elements is available, comprising, on the one hand, a base intended to be connected to the upper end of a tibia and defining a median antero-posterior axis and, on the other hand, a plurality of shoes each adapted to be fixed on the base and each defining a tibial prosthetic articulating surface intended to cooperate with a femoral prosthetic element, which articulating surface of each shoe presents both two glenoidal cavities for receiving internal and external condyles of the femoral element and an anterior spine which longitudinally defines an axis, on either side of which these glenoidal cavities are located, the different shoes of the set being adapted to be mounted on the base in respective angular positions in each of which the axis of the anterior spine of each shoe forms, with the median axis of the base, a predetermined angle which is possibly zero;
the upper part of a tibia is subjected to resection and there is connected to this resectioned end the base or a phantom base of the same geometry as the base to be implanted,
a shoe is chosen from the shoes of this set, and
the chosen shoe is fixedly mounted on the base, after having, if necessary, replaced the phantom base by the base to be implanted.

According to an advantageous characteristic of this process, in order to choose the shoe to be mounted from the shoes of the set, and after having connected the base to be implanted or the phantom base on the tibia:
there is mounted on this base a phantom shoe adapted to be retained in mobile manner on the base and defining a tibial prosthetic articulating surface intended to cooperate with the femoral anatomical or prosthetic element, which articulating surface of the phantom shoe presents an anterior spine defining a longitudinal axis,
the knee joint is entrained in at least one movement of flexion-extension,
the residual angular offset between the median axis of the base and the longitudinal axis of the anterior spine of the phantom shoe is determined, and
there is chosen, from the shoes of the set, the shoe whose predetermined angle is substantially equal or the closest to the value of the angular offset determined with the aid of the phantom shoe.

The invention will be more readily understood on reading the following description, given solely by way of example and made with reference to the drawings, in which:

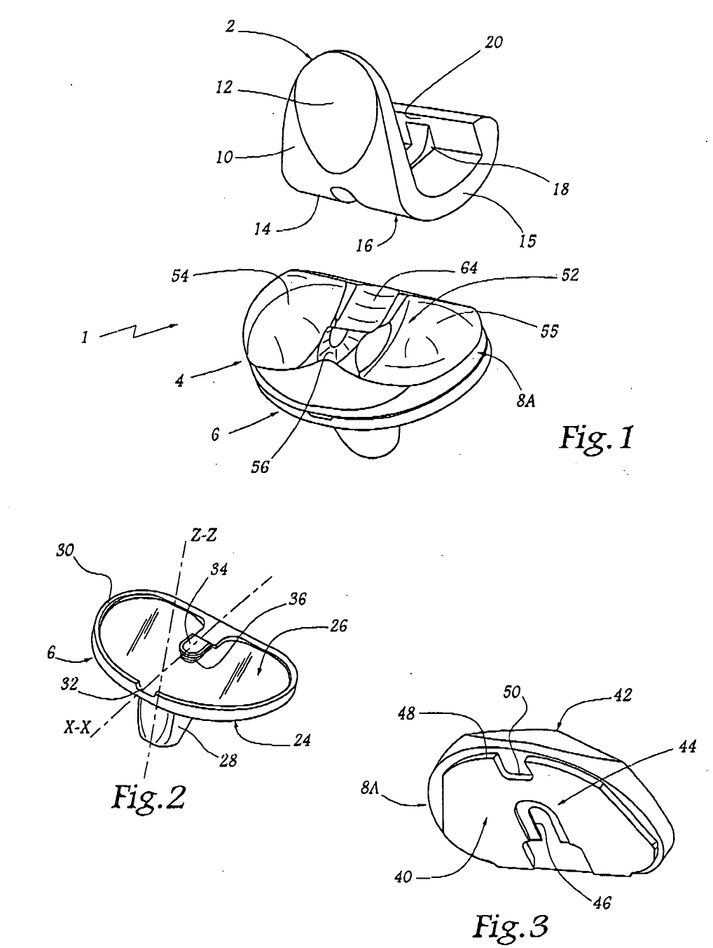

FIG. 1 is a view in perspective of a complete knee prosthesis comprising a tibial prosthetic assembly formed from a set of prosthetic elements according to the invention.

FIG. 2 is a view in perspective of the base of the tibial prosthetic assembly shown in FIG. 1.

FIG. 3 is a view in perspective, from an angle different from FIG. 2, of the shoe of the tibial prosthetic assembly shown in FIG. 1.

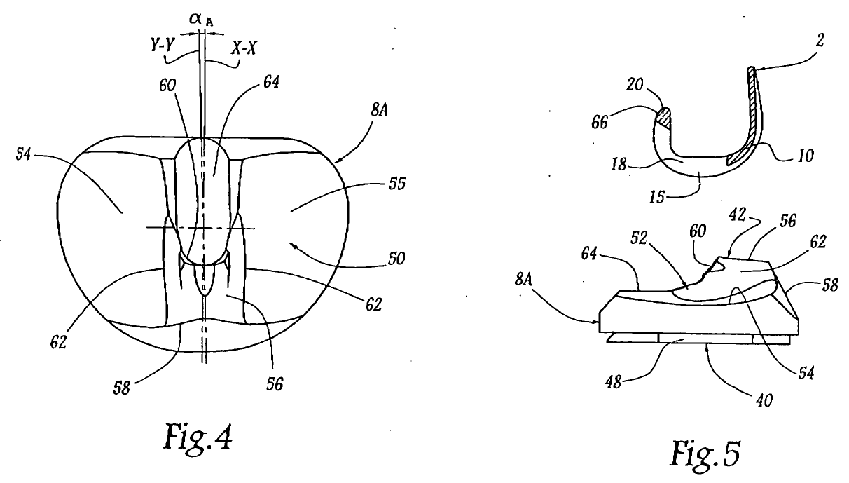

FIG. 4 is a view in elevation of the shoe of FIG. 3, taken in the direction of arrow IV indicated in FIG. 3.

FIG. 5 is a view in elevation, taken in the direction of arrow V in FIG. 4, of the shoe and a median section of the femoral element of FIG. 1, and

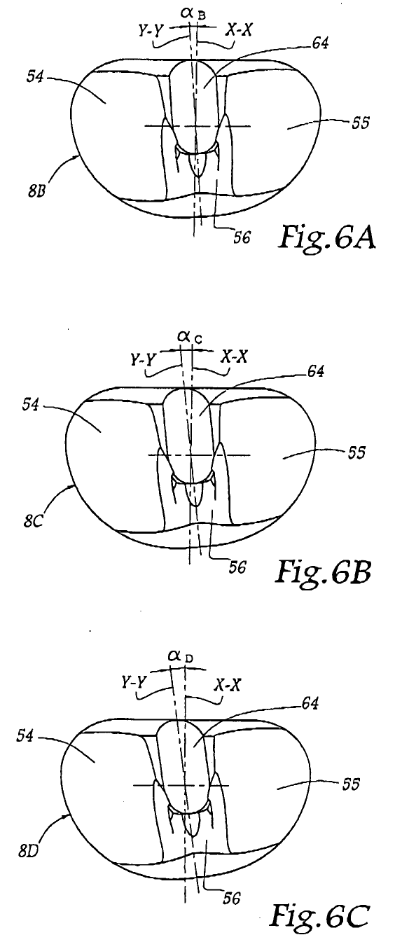

FIGS. 6A, 6B and 6C are views similar to FIG. 4 and respectively illustrate other shoes of the set of prosthetic elements according to the invention.

FIG. 1 shows a complete prosthesis 1 of a human knee joint. This prosthesis comprises, on the one hand, a femoral prosthetic element 2 intended to be connected to the lower end of a patient's femur, and, on the other hand, a tibial prosthetic assembly 4 comprising a base 6 intended to be connected to the upper end of the patient's tibia and a shoe 8A adapted to be fixedly mounted on the base. As will be explained in detail hereinafter, the shoe 8A issues from a series of a plurality of shoes.

In the following specification, the terms “anterior”, “posterior”, “internal” and “external” are understood to be meant in their anatomical sense, i.e. with respect to the femur or the tibia of the human skeleton when these bones are substantially aligned, i.e. in a position of extension of the knee joint.

More precisely, the femoral element 2 comprises an anterior plate 10 defining a front face 12 intended to cooperate with the patient's patella. The plate 10 is extended rearwardly by two internal (14) and external (15) condyles forming a femoral prosthetic articulating surface 16. The two condyles are separated from each other by a through groove 18 extending in an antero-posterior direction. The posterior end parts of the two condyles are connected by a bridge 20 which extends transversely to the longitudinal direction of the groove, forming the rear bottom wall thereof.

The base 6 of the tibial assembly 4, shown in greater detail in FIG. 2, comprises an essentially flat plate 22 whose lower and upper faces are respectively referenced 24 and 26. This plate defines an antero-posterior median axis X-X, the antero-posterior plane including this axis forming a plane of symmetry for the base.

On its lower face 24, the base 6 is provided with a tenon 28 extending along a longitudinal axis Z-Z substantially perpendicular to the plane of the plate 22. This tenon is intended to be introduced in the tibial medullary cavity and to be retained therein, for example by cement.

On its upper face 26, the base 6 comprises a projecting peripheral flange 30 interrupted in its front part by a notch 32. In its posterior part, the upper face 26 defines a projecting finger 34, extending longitudinally along axis X-X. The lateral edge of the finger is of rounded contour and defines a peripheral groove 36.

The shoe 8A, shown in greater detail in FIGS. 3 to 5, is a one-piece part made of plastics material, for example of polyethylene. It presents a lower face 40 and an upper face 42.

The lower face 40 is provided with means 44 for fixing the shoe on the base 6, adapted to allow the positioning and retaining of the shoe with respect to the base in fixed manner. As shown in FIG. 3, these means 44 comprise a lip 46 of rounded profile complementary of the profile of the finger 34, capable of being clipped in the groove 36 of the base 6. In addition, the exterior contour 48 of the lower face 40 is inscribed within the contour formed by the flange 30 of the base 6, the front part of the contour 48 being substantially complementary of the corresponding part of the contour of the flange 30. Consequently, once the shoe is placed in position on the base, the cooperation of shapes both between the finger 34 and the lip 46 and between the front parts of the exterior contour 48 and of the interior contour of the flange 30, ensures the immobility of the shoe 8A with respect to the base 6.

The lower face 40 of the shoe 8A comprises in its front part a notch 50 adapted to be disposed substantially in line with the notch 32 of the flange 30 when the shoe is placed in position on the base. By introducing the blade or the tip of an appropriate ancillary tool inside the notch 32 and the notch 50, it is possible to apply on the lower face of the shoe an upwardly directed lever effort in order to disconnect the shoe 8A from the base 6 by force. Such an operation intervenes in particular when the tibial assembly 4 is removed, for example at the end of life of this assembly.

The upper face 42 of the shoe 8A forms a tibial prosthetic articulating surface 52, shown in detail in FIGS. 4 and 5, intended to cooperate with the femoral articulating face 16. To that end, this surface 52 defines two principal glenoidal cavities 54 and 55, each intended to receive one of the internal (14) and external (15) condyles of the femoral element 2.

A projecting anterior spine 56 is formed between these two cavities. The spine 56 is intended to be housed in the groove 18 of the femoral element 2 so as to participate in the guiding of the articulating faces 16 and 52 with respect to each other.

This spine presents, in the antero-posterior direction, a longitudinal axis Y-Y forming an axis of symmetry for the glenoidal cavities 54 and 55. As indicated in FIG. 4, when the shoe 8A is mounted on the base 6, the axis Y-Y forms with the antero-posterior median axis X-X of the base 6 an angle noted α_{A}, worth about 2°.

The front face 58 of the spine forms a stop for the front bottom wall of the groove 18 separating the two condyles. In other words, this front surface 58 is intended to retain the femoral element 2 with respect to the tibial assembly 4 when the knee joint is stressed in hyper-extension.

As for the rear face 60 of the spine 56, it forms a stop surface for the bridge 20 connecting the two condyles 14 and 15. In other words, this rear surface blocks the femoral element 2 with respect to the tibial assembly 4 when the knee joint is stressed in hyper-flexion.

As for the lateral faces 62 of the spine 56, they limit the degree of freedom of rotation between the femoral element 2 and the tibial assembly 4, the geometry of these surfaces 62 being studied in order to allow the reproduction, which is as faithful as possible, of the corresponding anatomical movements.

At the rear and in line with the anterior spine 56, the tibular articulating surface 52 of the shoe 8A defines a third glenoidal cavity 64 intended to receive the posterior surface 66 of the bridge 20 which consequently forms a third femoral condyle. Depending on the concave profile of this cavity 64, the behaviour of the joint in flexion is rendered more faithful to the anatomical behaviour. For the shoe 8A shown, the axis Y-Y of the spine 56 forms an axis of symmetry for the glenoidal cavity 64.

As set forth hereinabove, the shoe 8A of FIGS. 1 and 3 to 5 belongs to a series of a plurality of tibial shoes essentially differing from one another by the value of the angle formed between the median axis X-X of the base 6 and the axis Y-Y of each shoe when these shoes are mounted on the base via fixing means 44. A set of prosthetic elements is formed by the base 6 and the series of a plurality of shoes of the type such as shoe 8A. More precisely, the set considered by way of example comprises both the shoe 8A and shoes 8B, 8C and 8D shown in FIGS. 6A to 6C, as well, possibly, as other shoes (not shown).

The lower faces of each of the shoes of the afore-mentioned set are substantially identical. They are all adapted to be individually mounted in fixed manner on the sole base 6 and all comprise to that end a clipping lip whose shape and orientation are identical to the lip 46 detailed for the shoe 8A. Only the exterior contour of these lower faces varies slightly from one shoe to the other, in order not to hinder the positioning of these faces inside the contour defined by the flange 30 of the base 6.

The upper faces of each of the shoes of the set comprise the same elements are those described for shoe 8A, namely two principal glenoidal cavities 54 and 55, an anterior spine 56 and a secondary glenoidal cavity 64. However, the overall arrangement of these elements varies from one shoe to the other: the axis Y-Y of each shoe 8A to 8D forms with the axis X-X of the base 6 an angle noted α_{A}, α_{B}, α_{C}, α_{D}, the respective values of these angles being about 2, 4, 6 and 8°.

The putting into place with dislocated patella of a tibial prosthetic assembly from the base 6 and the series of shoes 8A to 8D, is as follows:

After resection of the upper end part of the tibia and possible hollowing out of the internal walls of the tibial medullary cavity, the tenon 28 of the base 6, or in a variant a similar tenon of a phantom base of the same geometry as the base 6 subsequently implanted, is housed and rigidly immobilised in this medullary cavity, possibly being cemented.

The surgeon then places a phantom shoe capable of being retained on the base while conserving with respect to the latter at least one degree of freedom of rotation about an axis substantially parallel to axis Z-Z. This phantom shoe defines a tibial articulating surface similar to the articulating surface 52 detailed hereinbefore for the shoe 8A and presenting in particular an anterior spine similar to spine 56 for the shoe 8A. By entrainment of the knee joint in one or more movements of extension-flexion-extension, the surgeon provokes the displacement, particularly in rotation, of the phantom shoe with respect to the base. Once the knee is repositioned in position of extension, the surgeon measures the residual angular offset between the axis X-X of the base 6 and the longitudinal axis of the anterior spine of the phantom shoe, for example by means of graduations borne by the front edge of the shoe with respect to a mark borne by the front edge of the base. Depending on the patient's morphology, the pathology requiring the placing in position of a tibial prosthetic assembly and/or the positioning of the base with respect to the tibia effected by the surgeon, this residual angle may take any value included between zero and about fifteen degrees.

The surgeon then chooses from shoes 8A to 8D of the afore-mentioned set at his disposal, the shoe of which the angle of offset α_{A}, α_{B}, α_{C}, α_{D} is the closest to the angle measured, then, after having withdrawn the phantom shoe, places the chosen shoe in position on the base 6 via fixing means 44.

Thanks to the shoes of the set according to the invention, the surgeon is thus in a position to rapidly select the fixed shoe most appropriate for the patient in the course of operation. The cost of the tibial prosthetic assembly finally put into place is substantially identical to that of the heretofore used traditional assemblies, the shoes of the set not used being able to be stored and used for a subsequent intervention on another patient.

Although the set described hereinbefore comprises four shoes of which the respective offsets are worth 2, 4, 6 and 8°, it will be understood that the invention envisages sets comprising at least two shoes which present respective angular offsets different from one another, one of its shoes being able to present an axis Y-Y substantially aligned with the axis X-X of the base 6, i.e. present a substantially zero angle of offset.

Various arrangements and variants to the set described hereinabove may in addition be envisaged:
in order to reinforce the connection of the base 6 to the tibia, through holes may be provided in the plate 22 in order to receive screws for anchoring in the bone, these screws being placed in position from the upper face 26 of the plate before the shoe is mounted.
the lower surface 24 of the plate 22 may present an undulating profile, intended to promote the immobilisation of the base with respect to the tibia; and/or
the tenon 28 of the base 6 may be provided with a tapped orifice of axis Z-Z in order to allow the screwing of an added anchoring rod to be introduced more deeply in the tibial medullary cavity.

1. Set of prosthetic elements for a tibial prosthetic assembly (4), comprising a base (6) intended to be connected to the upper end of a tibia and defining a median antero-posterior axis (X-X), said tibial assembly comprising said base (6) and a shoe adapted to be fixedly mounted on the base (6) and defining a tibial prosthetic articulating face (52) intended to cooperate with a femoral prosthetic element (2), which articulating surface presents both two glenoidal cavities (54, 55) for receiving internal (14) and external (15) condyles of the femoral element (2) and an anterior spine (56) which longitudinally defines an axis (Y-Y) on either side of which are located these glenoidal cavities, characterized in that said set comprises a plurality of shoes (8A, 8B, 8C, 8D) adapted to be fixedly mounted on the base (6) in respective angular positions in each of which the axis (Y-Y) of the anterior spine (56) of each shoe forms, with the median axis (X-X) of the base, a predetermined angle (α_{A}, α_{B}, α_{C}, α_{D}), at least one of these predetermined angles being non-zero and another possibly being zero.

2. Set according to claim 1, characterized in that the shoes (8A, 8B, 8C, 8D) of the set are chosen from shoes of which the axes (Y-Y) of the spines (56) form with the median axis (X-X) of the base (6) angles (α_{A}, α_{B}, α_{C}, α_{D}) whose respective values are included between about 0 and 15°.

3. Set according to claim 2, characterized in that said angles (α_{A}, α_{B}, α_{C}, α_{D}) formed between the axes (Y-Y) of the spines (56) of the shoes (8A, 8B, 8C, 8D) of the set and the median axis (X-X) of the base (6) are respectively worth about 0, 2, 4, 6 and 8°.

4. Set according to claim 1, characterized in that each shoe (8A, 8B, 8C, 8D) of the set is provided with a face (40) for assembly opposite the articulating surface (52), said face bearing means (44) for fixing on the tibial base (6) adapted to cooperate with this base by complementarity of shapes.

5. Set according to claim 4, characterized in that the assembly faces (40) of the shoes (8A, 8B, 8C, 8D) of the set are substantially identical to one another.

6. Set according to claim 4, characterized in that the means (44) for fixing each shoe (8A, 8B, 8C, 8D) of the set comprises a lip (46) for clipping in a substantially complementary groove (36) borne by the base (6).

7. Set according to claim 4, characterized in that the base (6) comprises a peripheral flange (30) and in that the peripheral contour (48) of the assembly face (40) of each shoe (8A, 8B, 8C, 8D) of the set is adapted to be inscribed inside the flange (30) of the base (6).

8. Set according to claim 7, characterized in that at least corresponding parts of the interior contour of the flange (30) and of the peripheral contour (48) of the assembly face (40) of each shoe (8A, 8B, 8C, 8D) of the set are adapted to cooperate by complementarity of shapes as fixing means (44).

9. Set according to claim 1, characterized in that the articulating surface (52) of each shoe (8A, 8B, 8C, 8D) of the set presents a third glenoidal cavity (64) extending along the axis (Y-Y) of the anterior spine (56) and intended to receive a third condyle of the femoral element (2).

10. Process for putting in place a tibial prosthetic assembly (4) for a knee joint, in which:
a set of prosthetic elements is available, comprising, on the one hand, a base (6) intended to be connected to the upper end of a tibia and defining a median antero-posterior axis (X-X) and, on the other hand, a plurality of shoes (8A, 8B, 8C, 8D) each adapted to be fixed on the base (6) and each defining a tibial prosthetic articulating surface (52) intended to cooperate with a femoral prosthetic element (2), which articulating surface of each shoe presents both two glenoidal cavities (54, 55) for receiving internal (14) and external (15) condyles of the femoral element (2) and an anterior spine (56) which longitudinally defines an axis (Y-Y), on either side of which these glenoidal cavities are located, the different shoes (8A, 8B, 8C, 8D) of the set being adapted to be mounted on the base (6) in respective angular positions in each of which the axis (Y-Y) of the anterior spine (56) of each shoe forms, with the median axis (X-X) of the base, a predetermined angle (α_{A}, α_{B}, α_{C}, α_{D}) which is possibly zero,
the upper part of a tibia is subjected to resection and there is connected to this resectioned end the base (6) or a phantom base of the same geometry as the base to be implanted (6),
a shoe is chosen from the plurality of shoes (8A, 8B, 8C, 8D) of the set, and
the chosen shoe is fixedly mounted on the base (6), after having, if necessary, replaced the phantom base by the base to be implanted.

11. Process according to claim 10, in which, in order to choose the shoe to be mounted from the plurality of shoes (8A, 8B, 8C, 8D) of the set, and after having connected the base (6) to be implanted or the phantom base on the tibia:
there is mounted on this base a phantom shoe adapted to be retained in mobile manner on the base and defining a tibial prosthetic articulating surface intended to cooperate with the femoral anatomical or prosthetic element (2), which articulating surface of the phantom shoe presents an anterior spine defining a longitudinal axis,
the knee joint is entrained in at least one movement of flexion-extension,
the residual angular offset between the median axis (X-X) of the base and the longitudinal axis of the anterior spine of the phantom shoe is determined, and
there is chosen, from said shoes (8A, 8B, 8C, 8D) of the set, the shoe whose predetermined angle is substantially equal or the closest to the value of the angular offset determined with the aid of the phantom shoe.
